(19) United States
(12) Patent Application Publication
(10) Pub. No.: US 2006/0105010 A1
(43) Pub. Date: May 18, 2006

(54) Bladder implant

(76) Inventors: Martin Rahe, Huellhorst (DE); Rudi Maier, Engstingen (DE); Michael Wagener, Bremen (DE); Raymond Glocker, Aschffenburg (DE)

Correspondence Address: Karl F Milde jr;Milde & Hoffberg, 10 Bank Street, Suite 460, White Plains, NY, 10606, US

(21) Appl. No.: 10/530,383

(22) PCT Filed: Oct. 13, 2003
(86) PCT No.: PCT/DE03/03395

§ 371 Date: May 26, 2005

(51) Int. Cl. A61F 13/00; A61F 2/00

(52) U.S. Cl. 424/422; 424/423

(57) ABSTRACT

The invention prevents the occurrence of bladder infections by means of a silver-doped bladder implant that, upon placement at the entry of the bladder, results in the elimination of up to 95% of the germs arriving in the bladder via the urethra. The device not only prevents the occurrence of infection over a period of many months, but also enables the treatment of an existing infection. The device comprises a carrier adapted to be inserted and forming an implant body in the urinary bladder, and an active agent on the carrier having at least one of germicidal properties and properties that render germs ineffective to produce disease.

For many groups of patients, urinary bladder infections are a recurring problem. As rule, infections in the human bladder are caused by the migration of bacteria into the bladder space via the urethra. The urinary bladder itself is relatively immune to germs. The enormous problem with bladder infections is based on the risk of the infection ascending to the kidney via the ureter.

This problem is particularly great for persons suffering from incontinence or for older persons, as well as with traumatic changes in the central nervous system, which are accompanied by urination disorders. In many cases, these constellations lead to chronic urinary tract infections that, as a rule, require continuous medication, which, in turn, lead to progressive levels of resistance to antibiotics and finally to kidney problems.

Infections of the urinary tract collection system and of the organs processing urine are not only extraordinarily cost-intensive due to continuous need for medication and extended hospitalization, they are also very life-threatening. At the very least, they reduce significantly the quality of life, in particular when kidney damage leads to dialysis or to an implant of a donor kidney.

The constant increase of medications on the market, which are developed particularly for urinary tract infections, is a clear indication that these infections constitute a very great hazard.

It is the objective of the invention to limit the risk of infection already in the preliminary stages by eliminating the infectious germs that have entered via the urethra—see drawing Ref. 4—in the entry area of the bladder 3.

The enormous expenditures for treatment and the disadvantages of continuous prophylactic oral intake of medication that strains the entire body, in particular with long-term medication, are reduced by a significant amount. In this connection, it should be noted that with the typical use of antibiotics, therapies often require high overdoses, because the active ingredients are secreted in the urine in very different proportions and therefore do not fully arrive where they should actually be effective.

However, the decisive advantage of the solution subject to the invention is the fact there is no longer a need to use antibiotics, and therefore no resistant bacteria strains can come into existence.

The patient is kept free of urinary tract infections, and the treatment costs are reduced significantly. By reducing the risk of breeding resistance, one also receives a therapeutic reserve to an increased extent.

Thus, the main concept of the invention is to interrupt the movement of the germs to the ureters 2 directly into the bladder. In this manner, an ascending urinary tract infection can be avoided already in preliminary stages, without treatment using medication via the regular path of the blood circulation system or removing the germs by rinsing the bladder using a catheter. An additional aspect that needs to be mentioned with traditional treatment methods for urinary tract infections is the fact that a treatment can be started only after the germs have been diagnosed. In most cases, a prophylactic early detection is missing at this stage.

An implant that can be deposited in the bladder and that eliminates germs cannot only avoid an infection for a longer period but is also capable to treat an existing infection.

Realization of the Intravesical Implant—Prerequisites:

It is the objective of the invention to bring together an active agent carrier with specific properties with a suitable active agent that can be inserted into the bladder space as the implant, necessarily with a string-shaped geometry 12, via the urethra with a sufficient depot capacity.

In the actual bladder space, the implant shall—due to the higher temperature prevalent in that space—change from its initially stretched shape such that it will not be washed out, i.e., remain in the bladder.

So that no mechanical irritations would occur through bladder contractions at the roof and anterior wall of the bladder, the implant must be extremely flexible and soft.

With spastic urinary bladders, it may be necessary to prevent the contractions of the specific muscles through injections of Botulinum toxin.

Other conditions that the implant is to fulfill are absolute urine permeability as well as the necessity that no chemical or other adverse effects occur at the bladder periphery. In addition, it is important that the effectiveness is given for a long period—e.g., 6 to 12 months—and if possible, its expiration is indicated to the patient (e.g., using a dye).

An additional important requirement is that the implant can be removed at any time and in a simple manner.

All in all, numerous properties that can be realized by memory synthetics, among others, which are currently commercially available from several companies.

Realization of the Intravesical Implant—Achieving the Objective:

The objective of the invention is accomplished through the use of a respective synthetic polymer with the aforementioned memory properties, which fulfills the aforementioned conditions and that—as a matrix—is provided with a suitable germ-killing biocide such as nanosilver or magnesium oxide nanoparticles.

The Active Agent Carrier—Memory Polymer:

In this context, the so-called “shape memory” synthetics, which have been developed by Mnemoscience GmbH of Aachen, Germany, among others, may be used as the carrier.

These materials are capable of storing a pre-programmed shape and are, after deformation, restored automatically to exactly the same shape when applying a stimulus such as temperature, for example. It is possible to set the speed of the process.

In a preferred embodiment, the catheter shape 12 of the implant shown in FIG. 5 makes it possible to insert the elongated synthetic polymer directly into the urinary bladder using a lubricant.

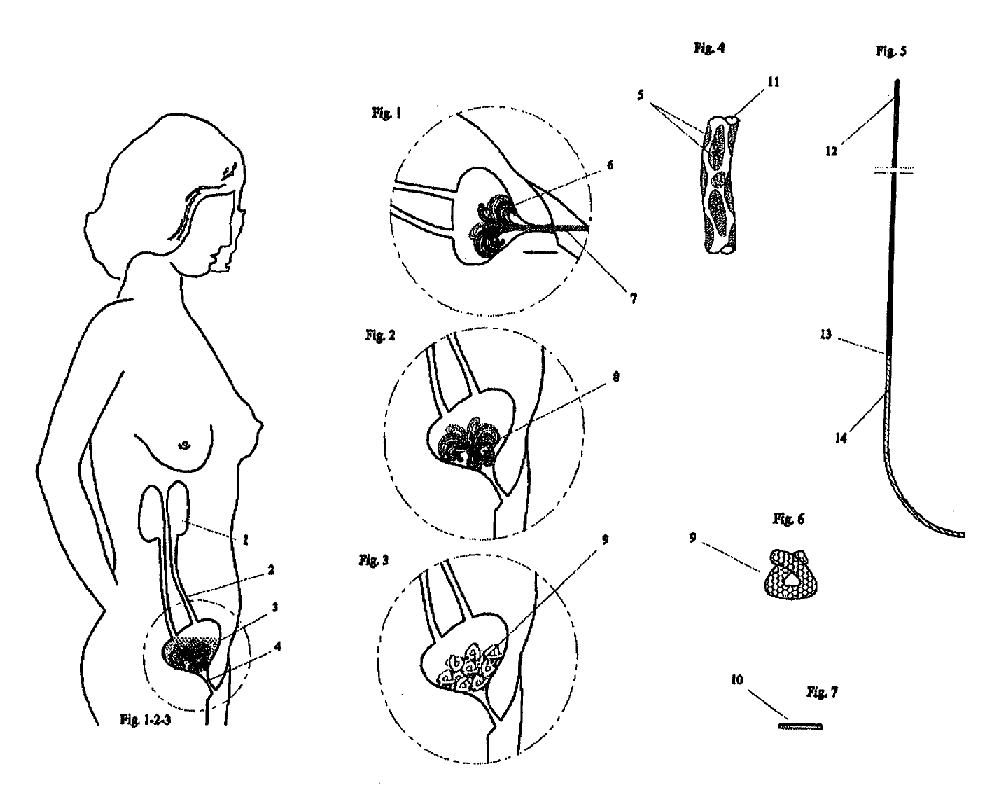

Triggered by the temperature change, the memory properties of the polymer string 12 lead to the string winding up like a ball of wool (not shown graphically), or separating into many extremely thin strings like a wad of cotton 6 (FIG. 1), such that it can no longer be rinsed out of the bladder outlet 4 and thus remain in the bladder for a long time.

Another variation of getting relatively large-volume implants through the urethra into the bladder is the option of foaming the synthetic material. In a compressed state 10 (FIG. 7)—at temperature state 1—the implant can be inserted minimal invasively into the urinary bladder via a catheter. By heating up to body temperature, the synthetic material remembers its original shape and assumes the programmed volume or shape 9 (FIG. 6) and thus its required large surface.

The implant shapes, illustrated in the drawings, that are only examples of many options, ensure a sufficient adaptive flexibility to the many volume changes of the urinary bladder and to the required permeability for urine. In addition, it must be fundamentally ensured that intermittent catheterization remains possible in spite of the implant.

An additional quality that distinguishes the polymer for the planned purpose is the option of using a special programming technology to specify whether the implant is to be biodegradable or biostable. Furthermore, the synthetic material can be set such that it dissolves or disintegrates enzymetically in the acidic range.

The Active Agent:

Metallic silver, magnesium oxide or respective substances in the form of colloids or nanoparticles that are incorporated as additives in the synthetic memory polymer form the foundation of the disinfecting properties of the implant.

The uniqueness of the properties of the “nanosilver”, which appears particularly well suited for the intended purpose, is initially preferred.

Studies prove that nanoparticular silver 5 (FIG. 4) has anti-microbial and fungicidal effects at very low concentrations (50 to 1000 ppm), without showing adverse health effects. The optimal properties of nanosilver for use as a biocide speak for a sensitive application such as that of an intravesical implant.

Viewed in detail, the silver ions released from the finely distributed silver act upon the microbes in three different ways: First, the silver ions metabolized by the microorganisms block the energy metabolism of the germs by disabling the sulfurous enzymes required for it.

Second, silver ions are distributed in the cells via the same channels as the essential calcium ions. In the cell itself, the Ag ions set a stop code at the DNA and in this manner prevent the reproduction of the microbes.

The third effect, which occurs through the compounding of the carrier materials with nanoparticular silver, is the fact that the bacterial adhesion is reduced significantly at hydrophilic surfaces. The germs no longer populate the object and thus the potential incrustation of the implant is initially avoided.

From a chemical standpoint, the optimal suitability of the nano-AG-technology for the purpose of the invention is based on the supply of a very big reservoir of effective silver ions without surplus and in a harmless concentration. As a fact, silver ions come into existence only when bacterial metabolic products come in contact with the metallic silver.

Regulation of the ion emission is carried out via a complex control circuit, where the primary control variables, the solubility of the silver salts that are generated on the surface, the wetting of the matrix polymer, which must have a sufficient size, and the corrosion of the metallic silver are important. This control circuit ensures that—with a sufficient contact area—metallic silver releases in a controlled manner only as many silver ions as are necessary for the re-generation of the silver compositions at issue. These, in turn, are dependent on the respective chemical environment. Specifically, if many germs are present, a relatively large amount of silver is metabolized. If no germs are present, no silver is used.

Through such a strictly controlled system, high silver ion concentrations are avoided and thus a germ-killing long-term effect is realized.

Since the implant will be used up over time, although slowly, because silver ions are continuously flushed out through the urine as salt of the bacterial metabolic products, it must be taken as given that after a certain time it will no longer be able to supply a sufficient amount of silver ions. Therefore, it must be replaced.

In this exemplary case, it is required that the active agent carrier be dissolved, for example by taking medication or through bladder irrigation with a suitable active agent (pH alteration, enzyme, etc.) and can thus be removed without the use of instruments.

What is claimed is:

1. A device for preventing and treating urinary bladder infections, comprising an active agent carrier, adapted to be inserted and forming an implant body in the urinary bladder, and an active agent on said carrier having at least one of germicidal properties and properties that render germs ineffective to produce disease.

2. A device as set forth in claim 1, wherein the active agent comprises particles in a colloidal form in a relatively uniform nano-size.

3. A device as set forth in claim 2, wherein the active agent is silver in a nano-porous state, which is introduced into the active agent carrier in a concentration of, for example, 0.1-2 percent in weight.

4. A device as set forth in claim 2, wherein the active agent silver in a nano-dispersive state is introduced into the active agent carrier in a concentration of, for example, 0.01-0.1 percent in weight.

5. A device as set forth in claim 3, wherein the active agent silver is introduced into the active agent carrier in a mixed manner in both the nanoporous and the nanodispersive state in a suitable ratio.

6. A device as set forth in claim 1, wherein the active agent is chemically bound to the active agent carrier as a matrix.

7. A device as set forth in claim 1, wherein the active agent carrier is a polymer that is equipped with shape memory properties or that obtains in the bladder space a shape suitable for the purpose through absorbing liquids, through temperature changes or through another stimulus.

8. A device as set forth in claim 7, wherein the active agent carrier dissolves, disintegrates or is changed into a different shape by changing the pH-value and/or in the presence of enzymes or another stimulus, such that it can be flushed out through the urethra together with the active agent.

9. A device as set forth in claim 8, wherein the active agent carrier is biodegradable, the duration of the disintegration being specified in the properties of the active agent carrier and its structure.

10. A device as set forth in claim 1, wherein the implant has an elongated shape such that it may be pushed into the bladder space directly or with the use of a catheter or another suitable device through the urethra or suprapubically.

11. A device as set forth in claim 10, wherein the elongated active agent carrier is comprised of many individual thin strings.

12. A device as set forth in claim 11, wherein, based on the shape memory properties of the active agent carrier material and the changed temperature, a ball or a wad is formed in the bladder space that can no longer be flushed out through the bladder outlet.

13. A device as set forth in claim 1, wherein the elongated active agent carrier is made of a foamed material in one piece or in individual shorter pieces, whereby during manufacturing the foam structure is pressed together such that the volume or the cross-section is relatively small prior to insertion in the bladder.

14. A device as set forth in claim 13, wherein the compressed active agent carrier foam returns to its previous foam structure in the bladder space due to its shape memory properties and the increased surrounding temperature and thereby assumes a relatively large volume.

15. A device as set forth in claim 14, wherein the implant body assumes a shape in the bladder space due to its shape memory properties, which on the one hand precludes a flushing out of the implant and on the other hand keeps the bladder outlet permeable to urine.

16. A device as set forth in claim 13, wherein in a water-soluble mechanically solid sheath that holds it firmly in its compressed shape.

17. A device as set forth in claim 16, wherein the implant body assumes its shape through the dissolution of the mechanically solid sheath and through the elasticity and/or through a swelling process in the bladder space.

18. A device as set forth in claim 1, wherein an end piece is attached to the implant body, which is equipped with a separable connection to the active agent carrier and that can be pulled out after insertion of the implant.

19. A device as set forth in claim 1, wherein the implant body is connected to a permanent catheter.

20. A device as set forth in claim 1, wherein the active agent is selected from the group consisting of silver, an antiseptic, an antibiotic and a biocide.
